(19) United States
(12) Patent Application Publication
(10) Pub. No.: US 2006/0015183 A1
(43) Pub. Date: Jan. 19, 2006

(54) Skeletal reconstruction device

(75) Inventors: Jonathan M. Gilbert, Marquette, MI (US); Qi-Bin Bao, Marquette, MI (US); Brian P. Janowski, Marquette, MI (US)

Correspondence Address: FITCH EVEN TABIN AND FLANNERY, 120 SOUTH LA SALLE STREET, SUITE 1600, CHICAGO, IL, 60603-3406, US

(73) Assignee: Pioneer Laboratories, Inc.

(21) Appl. No.: 11/177,989

(22) Filed: Jul. 8, 2005

Related U.S. Application Data
(60) Provisional application No. 60/587,072, filed on Jul. 9, 2004.

(51) Int. Cl. A61F 2/44

(52) U.S. Cl. 623/17.11

(57) ABSTRACT

A spinal column reconstruction device is disclosed including at least two bone surface engagement portions, each for fixation to distinct vertebral bone portions, a mobile portion of the device positioned between the two bone surface portions and providing for movement therebetween, and a motion limiter portion generally fixing the bone surface engagement portions in a predetermined positional orientation for reconstruction of the spine.

### CROSS-REFERENCE TO RELATED APPLICATIONS

This application claims the benefit of U.S. Provisional Application No. 60/587,072 filed Jul. 9, 2004, and entitled “SKELETAL RECONSTRUCTION DEVICE,” the specification of which is incorporated herein by reference in its entirety.

### DESCRIPTION

Several embodiments of a novel skeletal reconstruction implant are discussed and illustrated herein. The implant is generally comprised of a mobile portion, plate portions, and a motion limiter portion.

The primary embodiments of the implant are useful for spinal column reconstruction; however, the device can also be used for non-spinal orthopedic applications wherever it is desired to join two bones or bone parts. In spinal applications, the mobile portion of the device may be adjusted to assume different angles in the sagittal plane to restore the normal lordosis or kyphosis, and/or in the coronal plane to correct any scoliosis deformations. The device may also be adjusted along the longitudinal axis or transverse plane. Preferably, the mobile portion allows bone surface engagement portions on the plate portions to be oriented at any angle in all planes within the physiological spine curvature. Alternatively, the mobile portion may be restricted by the motion limiter portion to permit only selected motion, for example around a single plane or around a single axis with incremental change. In a preferred embodiment, the motion limiter portion may be formed to prevent or lock out generally all the motion otherwise allowed by the mobile portion. This complete locking mode is particularly useful when a fusion between bone portions is desired.

Relative motion occurring at the mobile portion provides for a multitude of possible angular and translatory orientations between a first bone surface engagement portion and a second bone surface engagement portion of the implant. This motion serves to feature the device as a variable angle or multi-positional reconstruction device. In preferred embodiments, this variation in angle or position is adjustable in-situ to match the unique physiological orientation of the bone portions within which the implant is located.

For example, if the device is used in the intervertebral space between two vertebrae, the upper plate portion and lower plate portion can be inserted first into the intervertebral space with the mobile portion orientating the bone surface engagement portions to match the unique curvatures of the vertebral bone portions of the patient's spinal column. The surgeon may then choose a motion limiter to lock the bone surface engagement portions in this orientation. For example, if the device is used in the patient's spinal column between the 4^{th} and 5^{th} lumbar vertebrae which has a 5 degree lordotic angle and 0 degree scoliotic angle between vertebral bone portions, the surgeon may choose a motion limiter wedge with these similar angular features to place and lock between the bone plate portions of the device. This motion limiting wedge will then serve to lock the implant in an orientation which matches the physiological spine curvature. Towards this end, the motion limiter may be formed such that it is interchangeable or removable.

There are instances when the surgeon may not wish to use a motion limiter to drive the implant to match the physiological space between the bone portions. Alternatively, the motion limiter may be used to drive a predetermined therapeutic angulation or orientation between the bone portions. For example, to reduce a problematic scoliosis, the surgeon may choose a motion limiting wedge which forces a reduced scoliosis. In any event, the bone surface engagement portions of the implant can be adjusted to a variety of orientations by the motion limiter portion.

The plate portion of the implant typically serves as a platform to seat features of the implant that interface with the bone portions and with the mobile and/or motion-limiting portions of the implant. The plate portion has a bone surface engagement portion for interfacing with a bone portion. The plate portion may also have a porous surface suitable for tissue in-growth such as bone tissue. Each bone surface engagement portion may include spikes, pegs, keels, or other bosses that protrude into the surface of the bone portions to be fused. The porous surface and protrusions assist in seating the plate portions into the exposed bone portion to prevent motion therebetween and for fixing the plate portion to the bone. For best fit, the bone surface engagement portions of the plate portion may be contoured or shaped to complement the shape of the bone portion with which it will mate. Therefore, the bone surface engagement portion may be flat, may be curved concavely or convexly, or may assume any other shape that complements with the bone portion.

A joint plate surface is located on a part of the plate portion that is not directly interfacing with the bone portion. This joint plate surface is shaped to complement or to be a component of the mobile portion of the implant. For example, the joint plate surface may have a concave profile to create a ball joint with a convex joint spacer or with an opposing convex surface formed on a separate plate portion.

The plate portions of each device may include apertures or other structure suitable for fasteners such as bone screws, or may include integrated fasteners, to secure the device to the surrounding bone mass of the bone portion.

The mobile portion of the implant may form several different kinds of joints. For example, the joint may be an articulating joint, such as a ball and socket joint, a hinge, or other variation of a mating concave-convex joint an elastomeric joint such as an elastomer situated between two plate portions, a pivot joint, a planar joint, or a joint incorporating a liquid or gas filled balloon, or any combination thereof. The motion occurring at the mobile portion provides for a multitude of possible angular and translatory orientations between the bone surface engagement portions of the implant.

Forms of the mobile joint may have a profile or configuration including concave, convex, or a combination of concave and convex, joint surface portions formed on each of the plate portions. A spacer portion of the implant, having convex or concave surface portions generally matching the curvature of the plate joint surface portions is formed on surfaces of the spacer portion. With the spacer portion situated between the two plates, two articulating joints are formed. The radii on the mating concave and convex joint surface portions may match or may be mismatched. If the radii are mismatched, the radius of the concave surface is generally larger than the radius of the convex surface, although either radius may be larger than the other. Another joint interface profile comprises a concave inner joint plate surface portion on one of the plate portions and a convex inner joint plate surface portion on the other plate portion. Joining these joint interface profiles forms an articulating joint between the two plates.

The motion limiter portion may be used to lock the bone surface engagement portions of the implant in a predetermined orientation or at least restrict their relative movement to a pre-determined range of motion. Depending on the type of motion limiter, it may be inserted or otherwise deployed before, during, or after implantation of the other portions of the device. The motion limiter may be integrated into a portion of the implant such as within the plate portion or spacer portion. It is preferred, however, that the motion limiter be a separate part so it can be applied at the surgeon's convenience after other portions of the implant are in place and the orientation between bone portions can be reevaluated.

The motion limiter may be in the form of a positional stop or lock. The stop portion is formed to block motion by the mobile portion of the implant. The final positional orientation of the bone surface engagement portions of the implant is then determined by the shape and/or position of the stop. The profile or configuration of a stop may vary, with some examples including a shaped insert such as a wedge, a sloped ring, a balloon filled with an incompressible material such as a curable polymer, a spring pin or otherwise deployable pin that springs into a predetermined recess to prevent movement between surfaces, a post or a cam which blocks the space within the mobile portion needed for motion to occur, or a plate which may support the plate portions in an immovable or limited-motion position. Such stops can be fixed or attached or mated to the implant in several different ways. Some examples include wedging, teeth engaging, screwing, snapping, camming, or locking into the implant. The stop may also be housed within the implant so as to not require separate attachment.

Another motion limiter or restrictor embodiment uses at least one strut placed in between the plate portions. The struts may be fixed or adjustable in length, and may be locked after a desired length is selected. The strut(s) may be connected to the plate portions to control the angulation of the plate portions and may be connected or otherwise fixed to the plate portions by a variety of methods. For example, screw threads or a ball and socket linkage may connect the strut(s) to the plate portion. If using screw thread, a variable angle screw head is preferred.

The motion limiter may also comprise an adhesive or other bonding agent such as calcium phosphate bone cement to lock the mobile portion in a pre-determined position. Such an agent may be used, for example, between the joint surface portions to bond them in fixed relation to each other. Similarly, along with several other biocompatible materials, the motion limiter portion may be made from bone or bone substitutes or other substances that enhance the growth of bone or provide a path for bone growth. One example is recombinant bone morphogenetic protein (BMP). By forming the motion limiter out of a suitable BMP, the material can enhance fusion and act as a motion limiting device. Alternatively, the motion limiter may be made from a collagen-based matrix.

The motion limiting portion of the implant may be made from a bioresorbable material such as a resorbable bone substitute or polymer. An example illustrating the benefit of this material occurs when the reconstructive device is used for delayed motion preservation. For example, it is often preferable after spinal reconstructive surgery that there is a period of immobilization at the surgical site during the early stages of healing. Therefore, it is beneficial to have an intervertebral motion preservation device, which is initially locked or limited in motion but will allow increased motion over time as healing progresses. By utilizing the bio-resorbable motion limiter, the reconstructive device can be implanted in a predetermined fixed or locked orientation. However, as the motion limiter is resorbed, the reconstructive device will regain a predetermined amount of motion and serve as a motion preservation device between the vertebral bone portions. Such a device can be used similarly at other joints of the body.

As a safety feature, the mobile portion of the implant may be designed to imitate a normal functioning intervertebral disc. Therefore, failure or absence of the motion limiter will not lead to complete implant failure. In this case, and in a backup mode of operation, the implant can adequately serve as a motion preservation device much like the normal human disc in the long or short term or until fusion across the implant occurs.

In the event the implant is used for multi-segment vertebral body replacement, each segment may include a mobile portion, so the motion or orientation of one vertebral body to the other at each segment can be adjusted.

Portions of the implant may be perforated or otherwise have passages to permit bone to grow into the implant as well as to grow through the implant from one bone portion to another. When present, these passages assist in obtaining optimal fusion between bone portions.

The implant can be manufactured from a variety of biocompatible materials. A non-exhaustive list of these materials includes PEEK and other biocompatible polymers, bone or bone substitutes, BMPs, stainless steel alloys, cobalt chrome, titanium and titanium alloys, or combination of these materials.

### DETAILED DESCRIPTION OF PREFERRED EMBODIMENTS

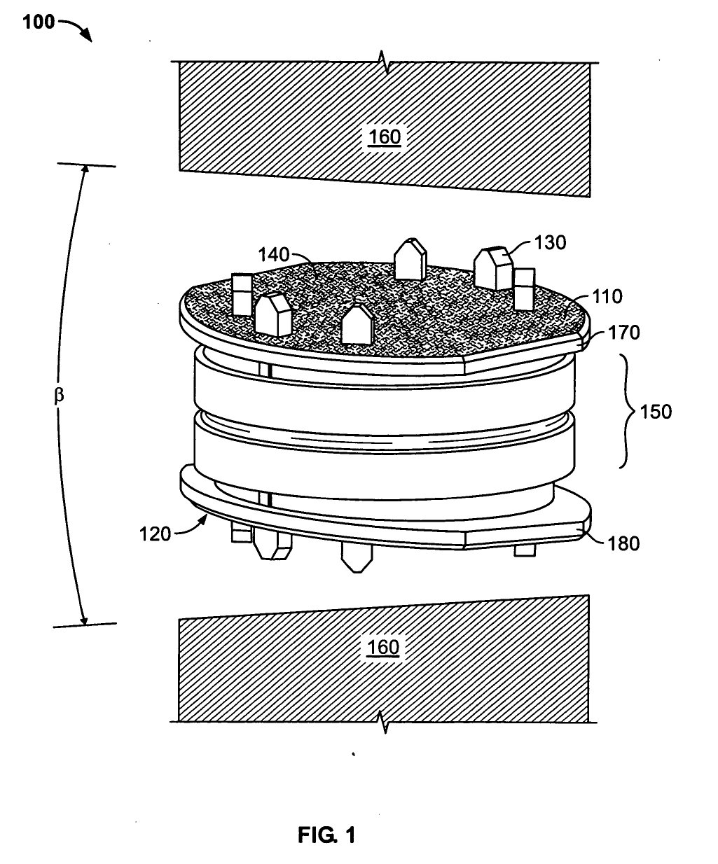

The embodiment in FIG. 1 shows a mobile portion 150 of implant 100. The mobile portion 150 is typically located between bone surface engagement portions 110, 120 and facilitates motion in at least one plane or around at least one axis between each bone surface engagement portion 110, 120. The motion provided by the mobile portion 150 of the implant 100 enables the bone surface engagement portions 110, 120 to orientate congruently to bone portions 160 to maximize the surface contact between the bone surface engagement portions 110, 120 and bone portions 160. Maximizing this surface to surface-contact favors greater boney in-growth into porous surfaces 140 and consequently stronger fixation between the bone surface engagement portions 110, 120 and the bone portions 160. Both the porous surfaces 140 and the bone surface engagement portions 110, 120 may be formed on upper and lower plate portions 170 and 180.

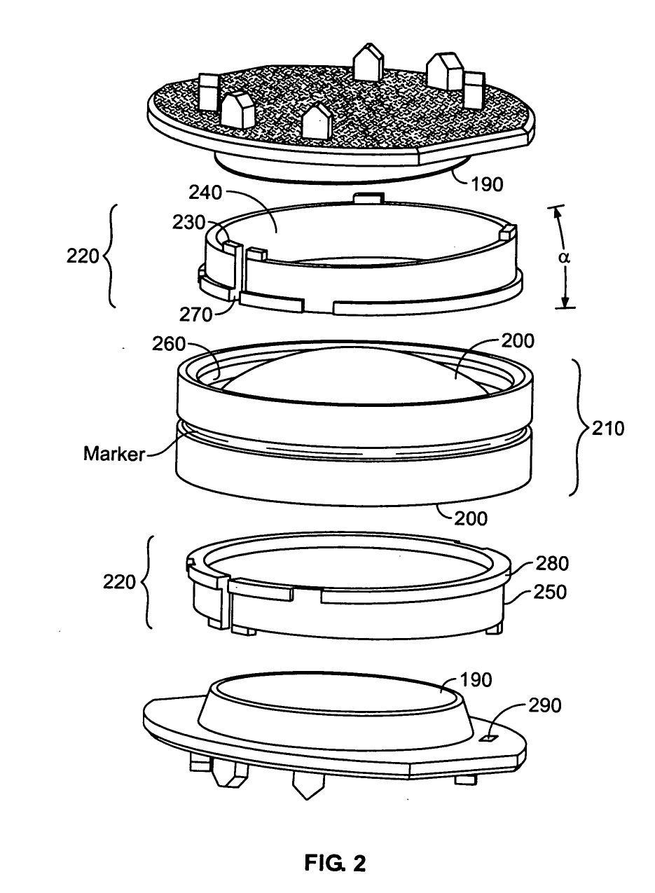

In the event the implant forms an articulating joint, the joint can have several different profiles or configurations. One embodiment of a spinal fusion implant 100 according to this invention (note FIGS. 1 and 2) comprises a first bone surface engagement portion 110 and a second bone surface engagement portion 120 for fixation to distinct bone parts. These bone surface engagement portions 110,120 are formed on the upper 170 and lower 180 plate portions. Joint plate surfaces 190 are also formed on the upper 170 and lower 180 plate portions, and complementing joint spacer surfaces 200 are formed on a joint spacer 210. In this embodiment, joint motion may occur bi-modally between each joint plate surface 190 and joint spacer surface 200.

The motion limiter portion 220 in this embodiment comprises an upper limiter 240 and a lower limiter 250. Although the limiters 240, 250 in this embodiment are ring shaped, it is noted that they can be of a multitude of profiles provided they ultimately support or fix or lock the plate portions 170, 180 at a desired orientation. As shown, these limiters 240, 250 may include a split wall 270, in this case, to allow elastic compression of the limiter 240, 250 before mating a retaining ridge 280 in a receiving groove 260 of the joint spacer 210. The split wall 270 is not necessary as the limiter 240, 250 and the retaining ridge 280 may be sized for a snap-fit into the receiving groove 260. The limiter 240, 250 may utilize many other types of connections such as a threaded connection, or a push and turn bayonet-type of connection. Also in this embodiment, note that the limiters 240, 250 may include locking tabs 230 and locking recesses 290, or other types of bosses or features, to prevent rotation or other movement of the limiters 240, 250 relative to other components of the device.

The motion limiters 220 are sloped at a preferred angle α (FIG. 2). A kit of this fusion device may include a plurality of interchangeable motion limiters 240, 250 each being sloped at a different value for the angle α. Such sloping may occur in more than one plane. The proper combination of sloped motion limiters 220 at varying predetermined angles enables the implant to best match the angle β between the bone portions 160. A kit may also include other components of the device in a variety of sizes and thicknesses as desired due to varying needs for different patients, and may include instruments for insertion of the implant. The spinal fusion implant 100 may be sized to adhere to all or part of the bone portions 160. In the case where the device is used for a spinal operation, the device may be sized to occupy all or part of an intervertebral space.

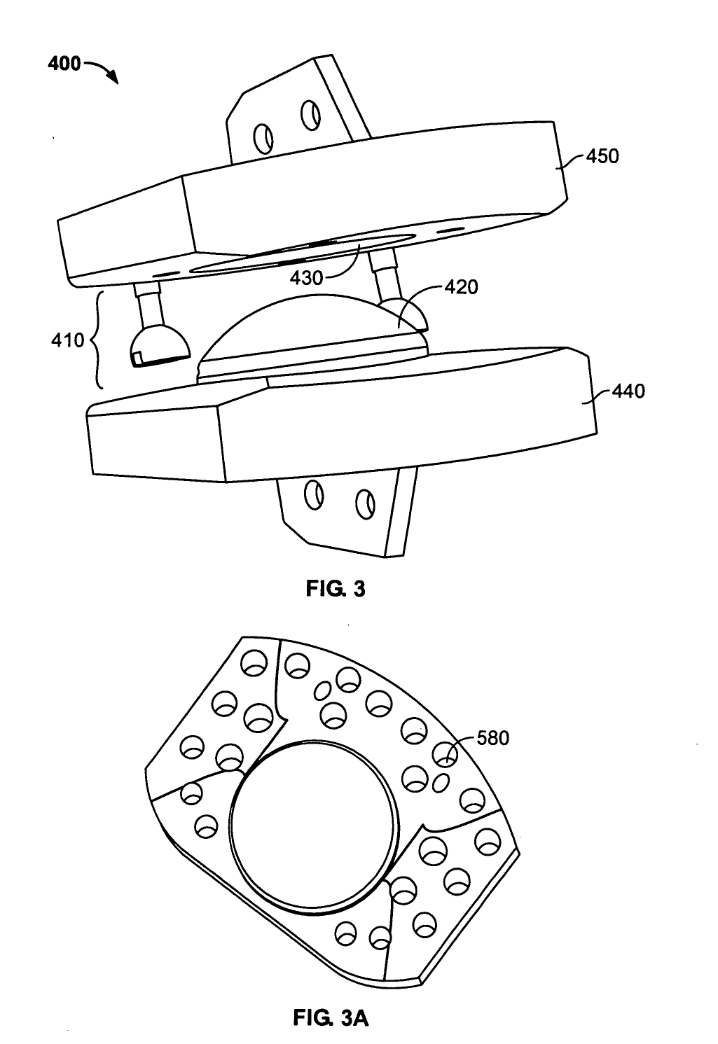

In an alternative articulating joint profile, the mobile portion 410 of an implant 400 may not have a spacer. In one such embodiment (FIG. 3), concave 430 and convex 420 joint plate surfaces are formed on an upper plate portion 450 and on a lower plate portion 440 of the implant. Although shown with the concave joint plate surface 430 on the upper plate portion 450, the joint plate surfaces 420, 430 may be reversed so that the convex joint plate surface 420 is formed on the upper plate portion 450 with the concave joint plate surface 430 being on the lower plate portion 440. Note that, as shown in FIG. 3A, all plate portions 440, 450 may include apertures, slots, or other passageways 580 for bone screws (not shown) or other fasteners for securing the plate portions 440, 450 against the bone portions. Note also that apertures, tunnels, or other passageways 580 may be used to promote fusion by opening a path for bone growth through the implant and between the bone portions. Although not shown in 3A, these passageways 580 may also extend through the joint plate surfaces of the implant. The passageways may be straight or curved, and may vary in diameter.

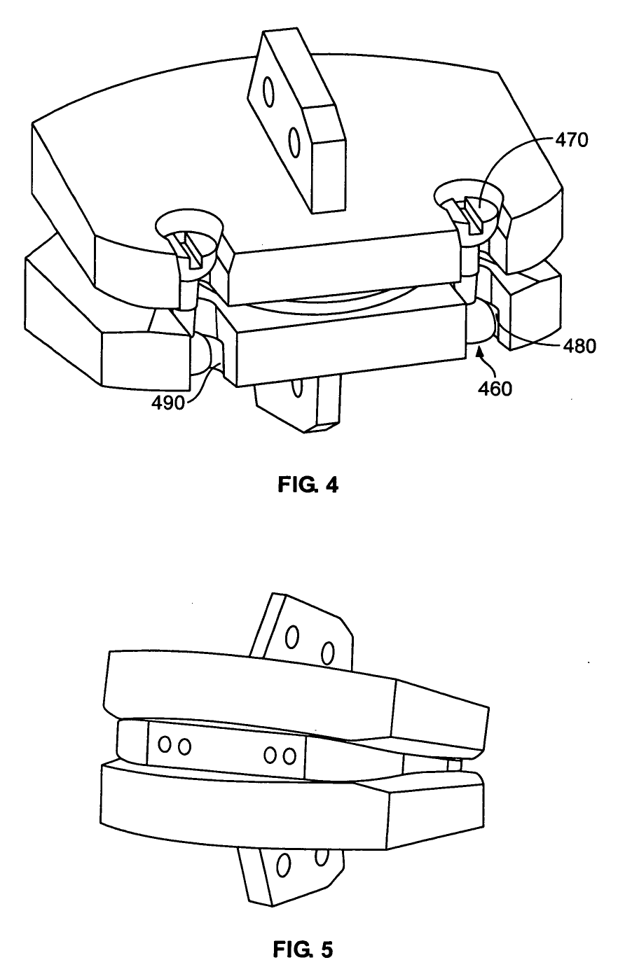

A similar embodiment to that of FIG. 3 is illustrated in FIG. 4 with the motion limiter portion 460. In this embodiment, the motion limiter 460 is shown as two fixation struts 470, although three fixation struts 470 located at generally three points of an isosceles triangle around the perimeter of the motion portion 410 are preferred. The fixation struts 470 may be either fixed or adjustable in length, as selected by the surgeon. If fixed, the user may select from a variety of lengths of fixed-length fixation struts. These struts may then be positioned within a strut access 480 of the plate portions 440, 450. To ease insertion, the fixation struts 470 may have a bayonet connection between their ends such that each end is pushed together, and a twist of 90 degrees, for example, will lock the fixation strut 470 ends together. It is preferred that the fixation struts 470 have a poly-axial head to articulate within a complementary shaped seat within the plate portions 440, 450. Examples of poly-axial heads are semi-spherical or chamfered head profiles. The fixation seat 490, on the other hand, may be chamfered, radiused, or have a single line contact formed by having a smaller through diameter the strut access 480 than for a diameter of the fixation seat 490. In any case, the fixation strut 470 and fixation seat 490 preferably cooperate to firmly support the fixation strut(s) 470 in the desired orientation. Fixation struts 470 that are variable in length can be used without the need for an assortment of fixed length fixation strut 470 sizes. As an example, the variable fixation struts 470 shown in FIG. 4 may be threaded together and relative rotation between the struts 470 threadably advances one relative to the other to shorten or lengthen their combined length. Similarly, the struts 470 may be in the form of a releasable plate (not shown) spanning between the plate portions 440, 450 to hold the plate portions 440, 450 in a predetermined orientation.

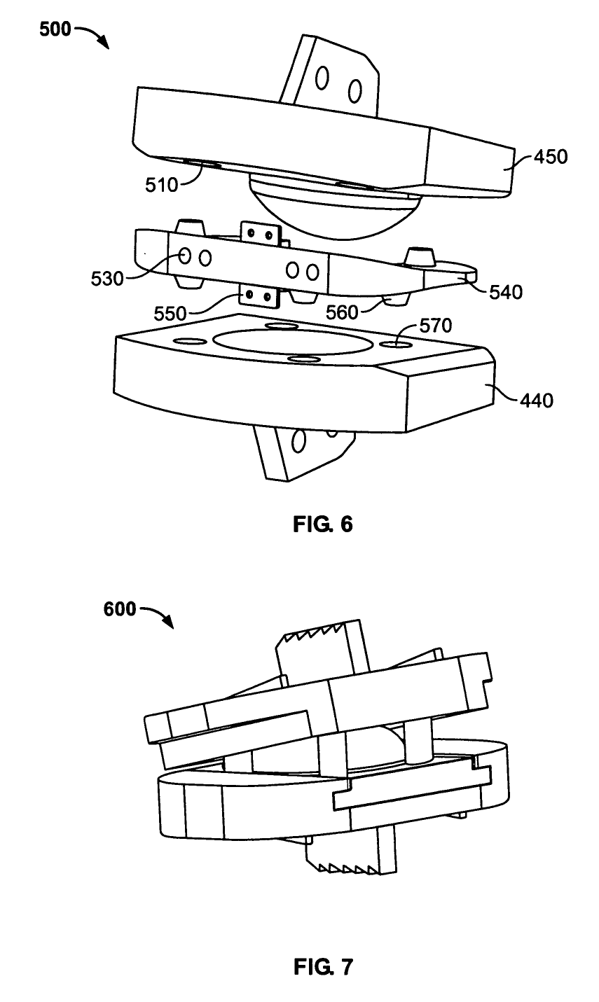

Yet another form of the motion limiter portion 500 is illustrated in FIGS. 5 & 6 in a preferred embodiment of the reconstructive device. In this embodiment, the upper and lower plate portions 440, 450 of the device are stabilized in a pre-determined orientation through the insertion of a positional wedge 540 between the plate portions 440, 450. This wedge 540 may come in a variety of pre-determined angulations in all planes and as with many of the motion limiter portions, it may be inserted after the plate portions 440, 450 or at the same time or along with the plate portions 440, 450. Assuming insertion after the plate portions 440, 450, the sloped shape of the wedge 540 eases insertion between the plates 440, 450 by driving the plate portions 440, 450 apart while concurrently sliding between them. Once in position, the positional wedge 540 is secured between the plate portions 440, 450 by common locking, fastening, or other attachment methods. For example, in the embodiment shown in FIG. 6, wedge-fastening holes 530 are provided in the positional wedge 540. These holes line up with complementing plate-fastening holes 510 situated on the upper and lower plate portions 440, 450. These holes 510 may be threaded and may house fasteners spanning between the positional wedge 540 and the plate portions 450, 440.

Alternatively, and as another example, the positional wedge 540 may have locking tabs 550 that span across the front of the upper and/or lower plate portions 450, 440. Fasteners or other connectors may be used to secure the locking tabs 550 to the plate portions 450, 440. As yet another example, the wedge 540 and/or the plate portions 440, 450 may include locking ridges, teeth, steps, bosses 560, locking recesses 570, or other features that interlock once the wedge is inserted between the plate portions 440, 450. The positional wedge 540 is particularly well-suited to be made from bone or a bone substitute due to its simple shape, and may be bioresorbable.

As discussed previously, a motion limiter may be a balloon filled with an incompressible or minimally compressible filler material such as a curable polymer. For example, the positional wedge 540 may be a wedge-shaped balloon. This balloon typically has an entry site that is punctured or has a valve to provide an entry for the inflating filler material. The preferred material of choice for balloon inflation is a curable polymer or bone cement, though it may be of any variety of fluids such as saline. It is preferred that a variety of sizes and angulations of balloons are provided. When filled, the balloon distends to a predetermined shape, thereby positioning the plate portions 440, 450 to a predetermined orientation. Alternatively, the plate portions 440, 450 may be first positioned in a desired orientation, followed by curing material in the balloon to retain this orientation. However, the orientation of the plate portions 440, 450 can be completed at any stage of implantation. The balloon is only one example of how the motion limiter may be inserted or otherwise deployed before, during, or after implantation of the plate portions and/or motion portions of the implant.

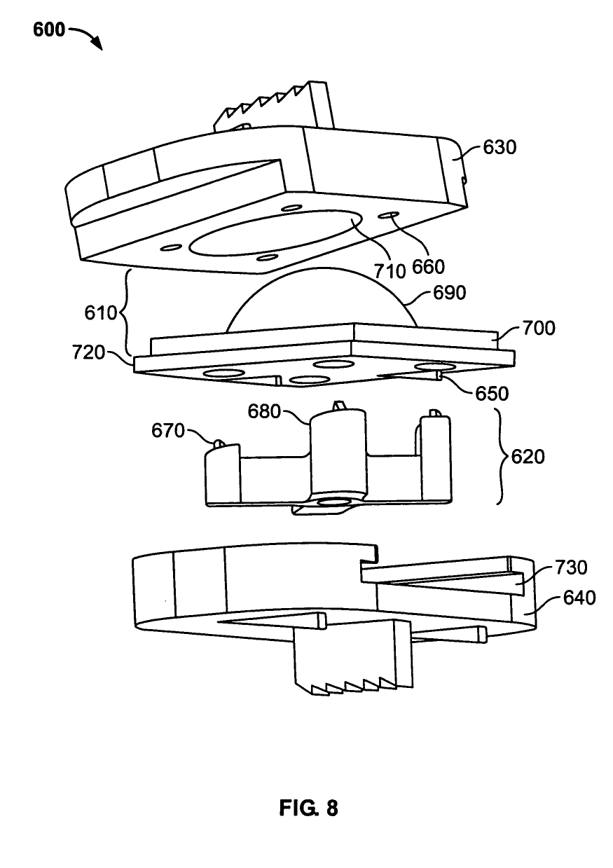

As another example of a device including an alternative articulating joint profile, one or more components of the joint surface portions may be formed on an insert that is slid into, attached, fixed or otherwise housed within a plate portion, such as device 600 shown in FIGS. 7 and 8. The device 600 has similar upper 630 and lower 640 plate portions. The mobile portion 610 of the implant includes an insert spacer 700, which has a joint insert surface 690 articulating with a joint plate surface 710. Insert spacer 700 may be inserted or otherwise held by one of the plate portions 630, 640. The insert spacer 700 may include features to secure the insert spacer 700 to the plate portion 640 such as one or more insert locking tab 650 that fall into a recess (not shown) in the lower plate portion 640. The insert spacer 700 may additionally include insert rails 720 while the lower plate portion 640 has complementing insert guides 730, though this configuration may be reversed. The insert spacer 700 may be sized and formed such that, although secured within the lower plate portion 640, the spacer 700 has some ability to slide within a plane along its generally flat bottom, thus adding additional degrees of freedom of motion between the plate portions 630, 640.

The motion limiter portion 620 of the device 600 includes angulation posts 680, which mate with the insert spacer 700. The motion limiter portion 620 is secured within the insert spacer 700 once the insert spacer 700 is slid into the lower plate portion 640. This is due to the angulation posts 680 protruding through the insert spacer 700 and the lower plate portion 640 blocking release of the insert spacer 700.

The motion limiter portion 620 may provide an angulation. Towards this end, the angulation posts 680 may be provided with a variety of slopes or angles and with differing heights, thereby providing a pre-determined and desired amount of angulation. At least one angulation post 680 preferably includes an anti-rotation tab 670 received in a locking recess 660 on the mating upper plate portion 630. Similar features common to preventing rotation between two bodies may be used.

For all embodiments, these bone surface engagement portions 110, 120 of the implant, regardless of whether they include protrusions, may have a porous surface 140 with porosity in the range of 100-1000 um for optimal bone in-growth into the implant. For example, the porous surface 140 may comprise a porous material such as porous nitinol or tantalum, a porous coating such as sintered metal particles, or other similar functioning material that the bone can grow into to assist in fixation of the implant 100 with the boney segment 160.

While there have been illustrated and described particular embodiments of the present invention, it will be appreciated that numerous changes, modifications, and combination of features will occur to those skilled in the art, and it is intended in the appended claims to cover all those changes, modifications, and combinations which fall within the true spirit and scope of the present invention.

1) A spinal column reconstruction device comprising:
at least two bone surface engagement portions each for fixation to respective vertebral bone portions;
a mobile portion positioned between said two bone surface engagement portions and providing for movement therebetween;
and a motion limiter portion generally to fix said bone surface engagement portions in a predetermined positional orientation for reconstruction of the spine.

2) The spinal column reconstruction device of claim 1 wherein each bone surface engagement portion is porous for ingrowth of tissue.

3) The spinal column reconstruction device of claim 1 wherein said bone surface engagement portions include protrusions for fixation of the device to said bone portions.

4) The spinal column reconstruction device of claim 1 further comprising a plate portion with passageways for bone ingrowth.

5) The spinal column reconstruction device of claim 1 further comprising a plate portion with passageways for a fastener for fixing the plate portion to one of bone portion.

6) The spinal column reconstruction device of claim 1 further comprising a plate portion having a joint plate surface formed thereon.

7) The spinal column reconstruction device of claim 1 further including an articulating joint.

8) The spinal column reconstruction device of claim 7 wherein said articulating joint comprises concave and convex surfaces.

9) The spinal column reconstruction device of claim 7 wherein said articulating joint is selected from one of a hinge, a pivot, or a planar joint.

10) The spinal column reconstruction device of claim 7 wherein said articulating joint comprises a ball and socket joint.

11) The spinal column reconstruction device of claim 1 further including an elastomeric joint.

12) The spinal column reconstruction device of claim 1 wherein said mobile portion comprises a balloon.

13) The spinal column reconstruction device of claim 1 wherein said mobile portion comprises a spacer or an insert.

14) The spinal column reconstruction device of claim 1 wherein said motion limiter portion may be deployed before, during, or after implantation.

15) The spinal column reconstruction device of claim 1 wherein said motion limiter portion comprises a positional stop or lock.

16) The spinal column reconstruction device of claim 15 wherein said positional stop or lock comprises a wedge or sloped ring.

17) The spinal column reconstruction device of claim 15 wherein said positional stop or lock comprises a balloon.

18) The spinal column reconstruction device of claim 15 wherein said positional stop or lock comprises fixed or adjustable struts, locking tabs, or plates.

19) The spinal column reconstruction device of claim 1 wherein said motion limiter portion comprises an adhesive or other bonding agent.

20) The spinal column reconstruction device of claim 1 wherein said motion limiter portion comprises bone or a bone substitute.

21) The spinal column reconstruction device of claim 1 wherein at least a portion of said implant is bioresorbable.

22) A spinal column reconstruction device for fusing bone portions comprising:
upper and lower plate portions with bone surface engagement portions formed thereon;
a mobile portion for positioning said plate portions in a predetermined orientation;
a motion limiter portion to lock the bone surface engagement portions of the device in said predetermined orientation.

23) The spinal column reconstruction device of claim 22 wherein said plate portions comprise passageways to permit bone to grow into or through the device from one bone portion to another.

24) The spinal column reconstruction device of claim 22 wherein said motion limiter portion is positioned between said plate portions.

25) The spinal column reconstruction device of claim 22 wherein said motion limiter is fixed by screws or locking teeth.

26) The spinal column reconstruction device of claim 22 wherein said motion limiter is removable, or is resorbable by the body.

27) The spinal column reconstruction device of claim 22 wherein said mobile portion comprises an insertable spacer positionable between said upper and lower plates.

28) A spinal column reconstruction device comprising:
upper and lower plate portions with at least respective bone surface engagement portions formed thereon for fixation to respective vertebral bone portions in an intervertebral space between two vertebrae;
a mobile portion positioned between said bone surface engagement portions and providing for movement therebetween;
and an interchangeable or removable motion limiter portion to fix generally said bone surface engagement portions in a predetermined orientation.
